(19) United States
(12) Patent Application Publication
(10) Pub. No.: US 2017/0202283 A1
(43) Pub. Date: Jul. 20, 2017

(54) WEARING SET CAPABLE OF PREVENTING OVERLAPPED ARTICLES FROM SLIDING PAST EACH OTHER

(71) Applicants: Chao-Mu Chou, Taipei (TW); Shiu-Yin CHENG, Taoyuan City (TW)

(72) Inventor: Chao-Mu CHOU, Taipei (TW)

(21) Appl. No.: 15/408,370

(22) Filed: Jan. 17, 2017

(30) Foreign Application Priority Data
Jan. 19, 2016 (TW) 105101603
Jan. 4, 2017 (TW) 106100213

(51) Int. Cl. A41D 27/00; A41B 1/08; A41D 1/06; A41B 11/08; A41D 19/00; A61F 13/08; A43B 23/07; D03D 1/00; D04B 1/24; A61F 13/00; A61F 13/10; A63B 71/14; A41B 11/00

(52) U.S. Cl. CPC A41D 27/00; A63B 71/145; A41B 1/08; A41D 1/06; A41B 11/08; A41D 19/0041; A41B 11/00; A43B 23/07; D03D 1/00; D04B 1/24; A61F 13/00004; A61F 13/104; A61F 13/08; A41D 2400/80; A41D 2500/10; A41D 2500/20

(57) ABSTRACT

A wearing set capable of preventing overlapped articles from sliding past each other, including a first wearing article having at least one first engaging effect zone, and a second wearing article having at least one second engaging effect zone; where one zone of the first engaging effect zone and the second engaging effect zone has a tangling-hairs fabric and the other zone has a textured-loops fabric, the tangling-hairs fabric including a plurality of tangling-hairs fibers protruding outwardly from a surface thereof, the tangling-hairs fibers having curled terminals or ball-shaped terminals; the textured-loops fabric including a plurality of fibriform loops on a surface thereof; and when the tangling-hairs fabric and the textured-loops fabric are overlapped to contact each other, the fibriform loops will be engaged or adhered with the tangling-hairs fibers to prevent the first wearing article from sliding relative to the second wearing article.

### BACKGROUND OF THE INVENTION

Field of the Invention

The present invention relates to a wearing set, especially to a wearing set capable of preventing overlapped articles from sliding past each other.

Description of the Related Art General wearing sets are for health purpose, social politeness, or exhibiting personal style or social status, so as to meet different human needs in different occasions. However, there are still many inconveniences in using conventional wearing sets, for example:

1. A top can be easily caused to slip out from inside of the waist of a pair of pants, or move away from the external surface of the waist of a pair of pants when the wearer stretches his/her body, thereby untidying the outfit.

2. The bottom surface of a sock tends to slide relative to a lining within a shoe, thereby making it hard for a pedestrian wearing socks and shoes to walk easily.

3. The inner glove and the outer glove of a composite glove tend to slide relative to each other, making it hard for a person to use the composite glove to grip an object.

4. A strip of bandage wrapping a joint tends to slip off.

5. Tight suit and compression sleeve tend to not perfectly suit a wearer's build, which not only makes it hard for the wearer to wear or remove them, but often causes them to slide out of position on the wearer.

The inconveniences of the conventional wearing sets mentioned above are mainly due to deficient friction between overlapping surfaces thereof. On the other side, conventional attaching devices capable of providing large friction are not suitable for the wearing sets because of the roughness and stiffness they usually possess. For example, when two counterparts of a hook-and-loop (velcro) fastener are installed on a sock and a shoe lining separately, the attachment thereof will give a stiff and prickling sensation for a wearer. Therefore, the fastening means of wearing sets is required to provide moderate friction.

To solve the problem of inconveniences caused by deficient friction or prickling sensation caused by attaching devices of large friction mentioned above, one conventional solution is to provide a polymer material of high friction coefficients for a wearing set to prevent two overlapping surfaces thereof from sliding relative to each other. For example, a layer of PU particles can be coated over the bottom surface of a sock to increase the friction between the sock and a shoe lining; a rubber braid can be installed on an inner surface of a waist of a pair of pants to increase the friction between a top and the pants; a button can be used to fix an inner sleeve with an outer sleeve of a two-layer jacket to enable the inner sleeve to move simultaneously with the outer sleeve; or a hook-and-loop can be used for fastening an inner hood with an outer hood of a two-layer jacket.

However, the above conventional solutions all have problems of stiff and prickling sensation and limited attachment area, and a novel design for wearing sets is therefore needed.

### SUMMARY OF THE INVENTION

The main objective of the present invention is to disclose a wearing set capable of preventing overlapped articles from sliding past each other, where at least one first engaging effect zone of a first wearing article can be engaged or adhered with at least one second engaging effect zone of a second wearing article by virtue of a tangling-hairs fabric and a textured-loops fabric, thereby preventing the first wearing article from sliding past the second wearing article.

In one embodiment, the first wearing article is a top, and the second wearing article is a pair of pants, and the present invention functions as preventing the lower part of the top from slipping out from inside of the waist of the pants, or preventing the lower part of the top from slipping out of place when attached on the external surface of the waist of the pants, thereby maintaining a tidy outfit.

In one embodiment, the first wearing article is a pair of leggings, and the second wearing article is a pair of pants, and the present invention functions as preventing the pants from slipping relative to the external surface of the waist of the leggings, thereby maintaining a tidy outfit.

In one embodiment, the first wearing article is an inner glove, and the second wearing article is an outer glove, and the present invention functions as preventing the inner glove from slipping relative to the outer glove, thereby facilitating a wearer's firmly gripping an object.

In one embodiment, the first wearing article is a strip of bandage for wrapping a hand, and the second wearing article is a boxing glove, and the present invention functions as preventing the boxing glove from slipping relative to the bandage, thereby ensuring that the boxing glove and the bandage are firmly attached to each other when in use.

In one embodiment, the first wearing article is a tight suit or a compression sleeve, and the second wearing article is a strip of bandage, and the present invention characterizes in that the bandage is used to assist the tight suit or the compression sleeve in properly compressing an upper limb or a lower limb.

In one embodiment, the first wearing article is a pair of socks, and the second wearing article is a pair of shoe linings, and the present invention functions as preventing the socks from sliding relative to the shoe linings, thereby providing a comfortable wearing experience.

In one embodiment, the first wearing article is a scarf, and the second wearing article is a top, and the present invention functions as preventing the scarf from slipping off the top, thereby increasing the stability and convenience of wearing the scarf on the top.

To attain the foregoing objectives, a wearing set capable of preventing overlapped articles from sliding past each other is proposed, including:

a first wearing article having at least one first engaging effect zone; and

a second wearing article having at least one second engaging zone;

wherein one zone of the first engaging effect zone and the second engaging effect zone has a tangling-hairs fabric and the other zone of the first engaging effect zone and the second engaging effect zone has a textured-loops fabric; the tangling-hairs fabric includes at least one meldable-staple-fibers spun yarn and has a plurality of tangling hairs protruding outwardly from a surface thereof, the tangling hairs having curled terminals or ball-shaped terminals formed by a singeing process; the textured-loops fabric includes at least one different-shrinkage-fibers yarn component and has a plurality of fibriform loops on a surface thereof, the fibriform loops being formed by a thermal contraction process; and when the tangling-hairs fabric and the textured-loops fabric are overlapped to contact each other, the fibriform loops will be engaged or adhered with the tangling hairs to prevent the first wearing article from sliding relative to the second wearing article.

Another embodiment of the present invention discloses a wearing set capable of preventing overlapped articles from sliding past each other, including:

a first wearing article having at least one first engaging effect zone; and

a second wearing article having at least one second engaging zone;

wherein one zone of the first engaging effect zone and the second engaging effect zone has a tangling-hairs fabric and the other zone of the first engaging effect zone and the second engaging effect zone has a textured-loops fabric; the tangling-hairs fabric includes at least one meldable-filament-fibers yarn and has a plurality of tangling hairs on a surface thereof, the tangling hairs having curled terminals or ball-shaped terminals formed by a peaching process and a singeing process; the textured-loops fabric includes at least one different-shrinkage-fibers yarn component and has a plurality of fibriform loops on a surface thereof, the fibriform loops being formed by a thermal contraction process; and when the tangling-hairs fabric and the textured-loops fabric are overlapped to contact each other, the fibriform loops will be engaged or adhered with the tangling hairs to prevent the first wearing article from sliding relative to the second wearing article.

### BRIEF DESCRIPTION OF THE DRAWINGS

FIG. 1 illustrates a first embodiment of the wearing set of the present invention.

FIG. 2a illustrates a first embodiment of the tangling-hairs fabric of the present invention.

FIG. 2b illustrates a second embodiment of the tangling-hairs fabric of the present invention.

FIG. 3a illustrates a partially enlarged view of a different-shrinkage-fibers yarn of the textured-loops fabric of the present invention.

FIG. 3b illustrates a first embodiment of the textured-loops fabric of the present invention.

FIG. 3c illustrates a second embodiment of the textured-loops fabric of the present invention.

FIG. 4 illustrates a second embodiment of the wearing set of the present invention.

FIG. 5 illustrates a third embodiment of the wearing set of the present invention.

FIG. 6 illustrates a fourth embodiment of the wearing set of the present invention.

FIG. 7 illustrates a fifth embodiment of the wearing set of the present invention.

FIG. 8 illustrates a sixth embodiment of the wearing set of the present invention.

FIG. 9 illustrates a seventh embodiment of the wearing set of the present invention.

FIG. 10 illustrates an eighth embodiment of the wearing set of the present invention.

### DETAILED DESCRIPTION OF THE PREFERRED EMBODIMENTS

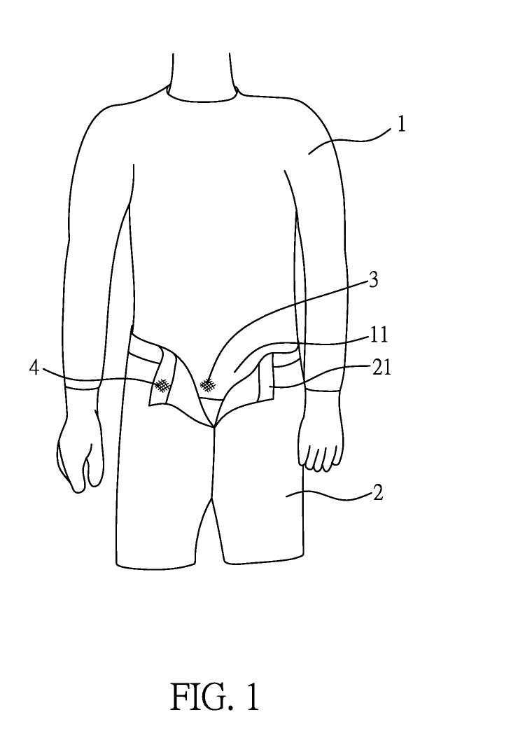

Please refer to FIG. 1, which illustrates a first embodiment of the wearing set of the present invention, the wearing set including a first wearing article 1 (a sport tight top, for example), and a second wearing article (a pair of sport tight pants, for example). The first wearing article 1 has at least one first engaging effect zone 11 implemented with a tangling-hairs fabric 3 on an external surface of a lower part of the sport tight top. The second wearing article 2 has at least one second engaging effect zone 21 implemented with a textured-loops fabric 4 on an inner surface of a waist of the sport tight pants.

When a user wears the first wearing article 1 and the second wearing article 2 and makes the first engaging effect zone 11 in contact with the second engaging effect zone 21, the tangling-hairs fabric will be engaged and adhered with the textured-loops fabric 4 to prevent a lower part of the sport tight top (the first wearing article 1) from slipping out from inside of the waist of the sport tight pants (the second wearing article 2) when the user exercises his/her body, thereby keeping a tidy outfit.

In an alternative embodiment, the first engaging effect zone 11 can be implemented with the textured-loops fabric 4, and the second engaging effect zone 21 can be implemented with the tangling-hairs fabric 3.

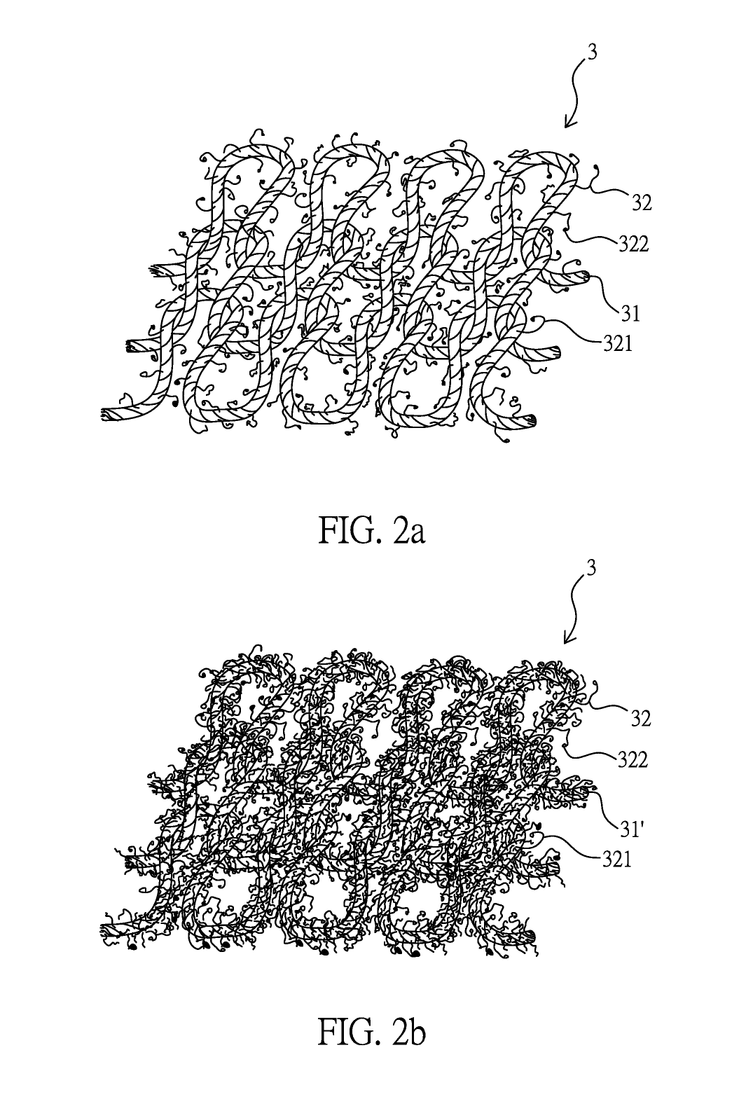

Please refer to FIG. 2a, which illustrates a first embodiment of the pile fabric 3, where, the tangling-hairs fabric 3 includes at least one meldable-staple-fibers spun yarn 31, and can be fabricated by a weaving process, a warp knitting process, or a weft knitting process. The tangling-hairs fabric 3 can include elastic fibers and can be implemented by the warp knitting process or the weft knitting process to be in the form of a single jersey mesh fabric, a single jersey fabric, or a double jersey fabric, or implemented by the weaving process to be in the form of a plain fabric or a twill fabric. The meldable-staple-fibers spun yarn 31 is fabricated by twisting meldable staple fibers with a spinning machine, with hairs formed by the terminals of the meldable staple fibers protruding out the surface of the meldable-staple-fibers spun yarn 31. As the hairs are randomly distributed over the surface of the meldable-staple-fibers spun yarn 31, only a small portion of the hairs will be protruding out the surface of the tangling-hairs fabric 3, so it is preferable to use a ring spinning process, which can produce more hairs, to prepare the meldable-staple-fibers spun yarn 31.

To offer a better fastening effect, the tangling-hairs fabric 3 can go through a singeing process to provide tangling-hairs fibers 32 having curled terminals 321 and/or ball-shaped terminals 322. As the tangling-hairs fabric 3 is implemented with the meldable-staple-fibers spun yarn 31, a strike-out process (for example, a peaching process or a brushing process) for producing more hairs can be omitted.

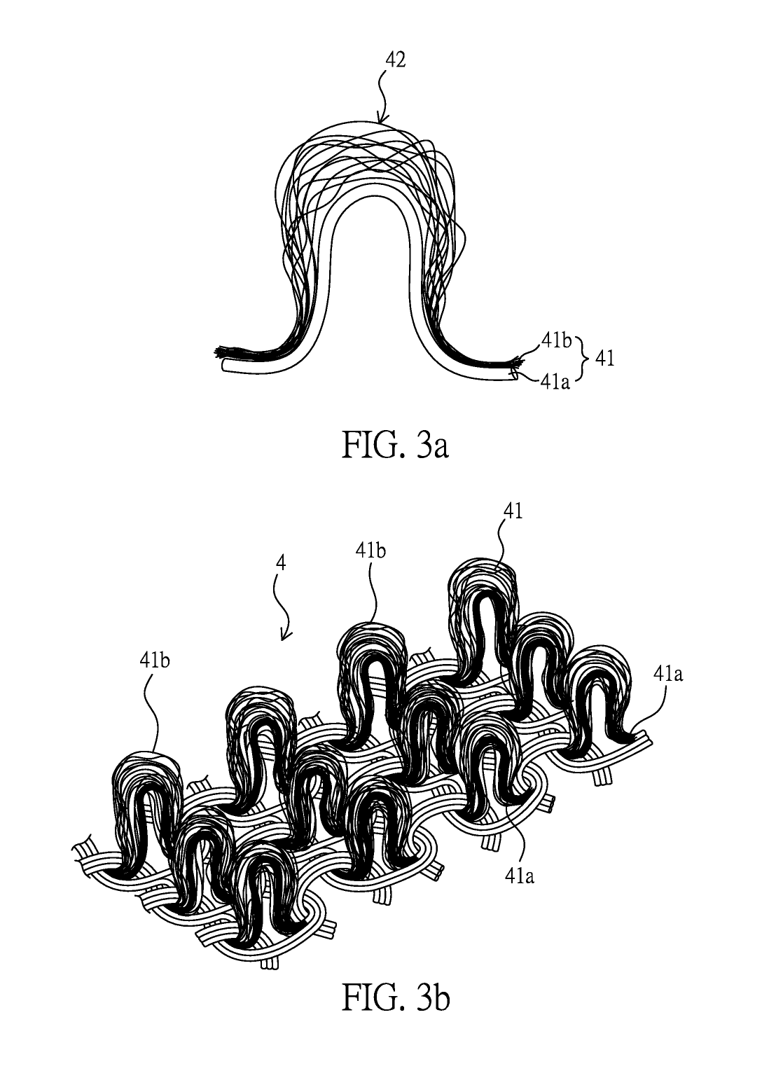

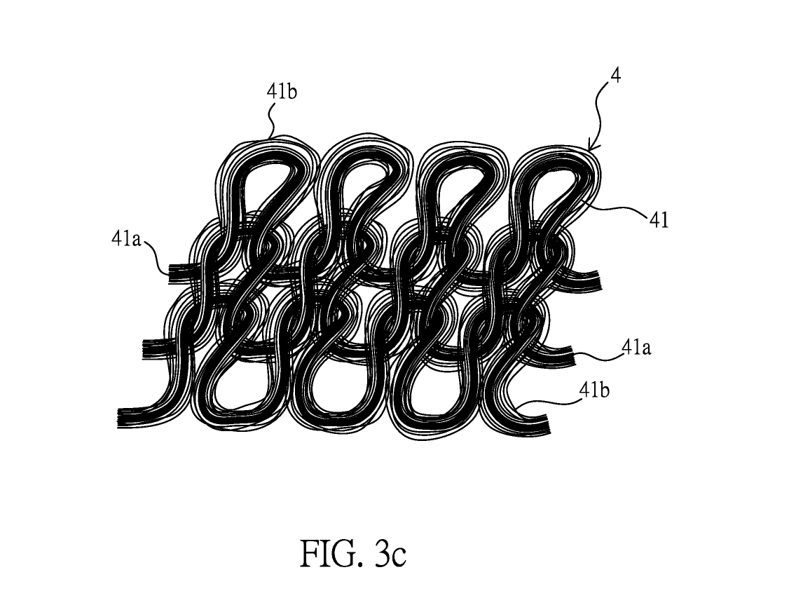

The meldable-staple-fibers spun yarn 31 can include wool or infrared-fibers to further provide a heat retaining function. Besides, the meldable-staple-fibers spun yarn 31 can be implemented with a yarn count of 10's to 80's, and the meldable staple fibers can be derived from uniformly cut filament fibers of 75 de/72 f, 100 de/72 f, 100 de/36 f, 150 de/48 f, 150 de/36 f, or 450 de/96 f, and the single fiber size thereof can be from 1.04 de/f to 4.69 de/f to match the size of fibriform loops 42 (shown in FIG. 3a) of the textured-loops fabric 4, thereby offering a perfect fastening effect. It is to be noted that fiber size is not limited thereto.

Besides, the meldable staple fibers can be combined with at least one kind of natural staple fibers to form a blended yarn to provide additional functions. As the natural staple fibers will be burned out during the singeing process and only the meldable staple fibers will be left in the tangling-hairs fabric 3, therefore, the meldable staple fibers are preferably to have a weight fraction more than 50% in the blended yarn to provide sufficient quantity of the tangling-hairs fibers 32.

The meldable staple fibers of the meldable-staple-fibers spun yarn 31 can be polyester fibers, polyamide fibers, polyolefin fibers, or polyacrylonitrile fibers, where the meldable staple fibers can be hollow fibers and/or have micro pores over fiber surfaces thereof, so as to increase the surface areas and thereby make the tangling-hairs fabric 3 capable of absorbing moisture and drying quickly. Besides, the meldable-staple-fibers spun yarn 31 can go through a sanitizing process or be twisted with silver-coated fibers to further provide an antibacterial effect; or the meldable-staple-fibers spun yarn 31 can be combined with metal fibers, such as silver-coated fibers, copper fibers, or graphene fibers, to provide an anti-electrostatic effect or an electric conduction function.

Please refer to FIG. 2b, which illustrates a second embodiment of the tangling-hairs fabric 3, where, different from the first embodiment, the tangling-hairs fabric 3 includes at least one meldable-filament-fibers yarn 31′, and a fabrication method thereof includes a strike-out process (for example, a peaching process or a brushing process) and a singeing process, so as to provide tangling-hairs fibers 32 having curled terminals 321 and/or ball-shaped terminals 322 and protruding from a surface of the tangling-hairs fabric 3. As the tangling-hairs fibers are formed by the strike-out process, the tangling-hairs fibers will emerge only on the peached or brushed surface of the tangling-hairs fabric 3, so by adjusting the processing time, using a proper kind of brush or abrasive paper, the quantity and length of the tangling-hairs fibers can be determined accordingly to offer a variety of fastening strengths for the tangling-hairs fabric 3, thereby fulfilling the needs of different applications.

Similarly, the meldable filament fibers of the meldable-filament-fibers yarn 31′ can be polyester fibers, polyamide fibers, polyolefin fibers, or polyacrylonitrile fibers. For example, the meldable filament fibers can be derived from a polyester textured yarn of a size of 75 de/72 f, 100 de/72 f, 75 de/36 f, 100 de/36 f, 150 de/48 f, 150 de/36 f, or 450 de/96 f, and the single fiber size thereof can be from 1.04 de/f to 4.69 de/f to match the size of fibriform loops 42 (shown in FIG. 3a) of the textured-loops fabric 4, thereby offering a perfect fastening effect, and it is to be noted that fiber size is not limited thereto.

The meldable filament fibers can be hollow fibers and/or have micro pores over fiber surfaces thereof, so as to increase the surface areas and thereby make the tangling-hairs fabric 3 capable of absorbing moisture and drying quickly. Besides, the meldable-filament-fibers yarn 31′ can go through a sanitizing process or be twisted with silver-coated fibers to further provide an antibacterial effect; or the meldable-filament-fibers yarn 31′ can be combined with metal fibers, such as silver-coated fibers, copper fibers, or graphene fibers, to provide an anti-electrostatic effect or an electric conduction function, where the sanitizing process is well known in the field and will not be further addressed.

The textured-loops fabric 4 can be fabricated by a weaving process, a warp knitting process, or a weft knitting process. The textured-loops fabric 4 can include elastic fibers and can be implemented by the warp knitting process or the weft knitting process to be in the form of a single jersey mesh fabric, a single jersey fabric, or a double jersey fabric, or implemented by the weaving process to be in the form of a plain fabric or a twill fabric. The textured-loops fabric 4 includes at least one kind of different-shrinkage-fibers yarn 41, where two different kinds of fibers having two different thermal contraction coefficients are twisted or combined by an air-texturizing process, and then go through a thermal contraction process to produce a fluffy effect by virtue of the difference of the two thermal contraction coefficients. The different-shrinkage-fibers yarn 41 includes first fibers 41a having a first thermal contraction coefficient preferably above 15%, where the first fibers 41a can be polyester fibers of a spec of 30 de/12 f, 40 de/12 f, or 50 de/24 f; and second fibers 41b having a second thermal contraction coefficient preferably about 3%, where the second fibers 41b can be segmented-pie type PET/Co-PET composite polyester fibers of a spec of 80 de/72×12 f, or islands-in-the-sea type composite polyester fibers of a spec of 75 de/24×37 f, or polyester fibers of a spec of 50 de/144 f or 75 de/72 f. The single fiber size of the second fibers 41b can be from 0.09 de/f to 1.04 de/f to match the size of the tangling-hairs fibers 32, thereby offering a perfect fastening effect. It is to be noted that fiber size is not limited thereto.

The segmented-pie type 80 de/72×12 f PET/Co-PET conjugated polyester fibers and the islands-in-the-sea type 75 de/24×37 f conjugated polyester fibers both need to go through a fiber-splitting process. For example, using a hot alkaline solution to provide a saponification effect to dissolve the conjugated polyester fibers of the second fibers 41b to derive fibers of a size from 0.09 de/f to 0.08 de/f, and thereby form the fibriform loops 42. As the different-shrinkage-fibers yarn 41 will begin to have thermal reaction at temperatures above about 50° C., therefore, the fiber-splitting process or a dyeing process thereafter can be utilized to simultaneously perform a thermal-contraction process on the different-shrinkage-fibers yarn 41.

Please refer to FIG. 3a, which illustrates a partially enlarged view of a different-shrinkage-fibers yarn 41, where, after the thermal-contraction process, the first fibers 41a shrink more than the second fibers 41b, making the first fibers 41a much shorter than the second fibers 41b and thereby enabling the second fibers 41b to form fluffy fibriform loops 42.

As mentioned above, to attain a perfect fastening effect, the tangling-hairs fibers 32 of the tangling-hairs fabric 3 and the fibriform loops 42 of the textured-loops fabric 4 have to match each other in size. For example, when the single fiber size of the fibriform loops 42 is smaller than 0.5 de/f, the single fiber size of the tangling-hairs fibers 32 is preferably larger than 1.0 de/f; and when the single fiber size of the fibriform loops 42 is between 0.5 de/f and 1.04 de/f, the single fiber size of the tangling-hairs fibers 32 is preferably larger than 2.0 de/f According to current state of art, as 50 de/144 f fibers can be produced without the need of going through a conjuated spinning process including a fiber-splitting process, the manufacturing cost can therefore be reduced in some applications of the present invention. As a result, different sizes of fibers can be fabricated by using different spinning process (with/without the fiber-splitting process) to meet the needs of different fastening effects in different applications of the present invention.

In addition, as the fastening effect of the present invention is generated by a friction force between the tangling-hairs fibers 32 and the fibriform loops 42, therefore, the friction coefficient between the tangling-hairs fabric 3 and the textured-loops fabric 4 must be proper designed for practical needs. After experiments, a preferable friction coefficient is found to be above 1.0.

Please refer to FIG. 3b, which illustrates a first embodiment of the textured-loops fabric 4, where the textured-loops fabric 4 is a woven fabric or knitted fabric having protruding pile-loops. The protruding pile-loops include at least one different-shrinkage-fibers yarn 41 processed by a thermal-contraction process, the second fibers 41b of the different-shrinkage-fibers yarn 41 forming the fibriform loops 42.

Please refer to FIG. 3c, which illustrates a second embodiment of the textured-loops fabric 4, where the textured-loops fabric 4 is a plain fabric implemented by a weaving process, a warp knitting process or a weft knitting process. The textured-loops fabric 4 includes at least one different-shrinkage-fibers yarn 41 processed by a thermal-contraction process, the second fibers 41b of the different-shrinkage-fibers yarn 41 forming the fibriform loops 42.

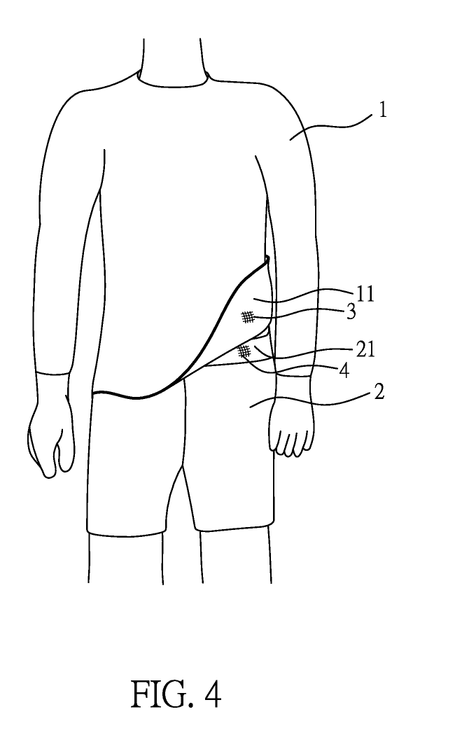

Please refer to FIG. 4, which illustrates a second embodiment of the wearing set of the present invention, including a first wearing article 1, which is a sport tight top, and a second wearing article 2, which is a pair of sport tight pants. The first wearing article 1 has at least one first engaging effect zone 11 implemented with a tangling-hairs fabric 3 on an inner surface of a lower part of the sport tight top. The second wearing article 2 has at least one second engaging effect zone 21 implemented with a textured-loops fabric 4 on an external surface of a waist of the sport tight pants.

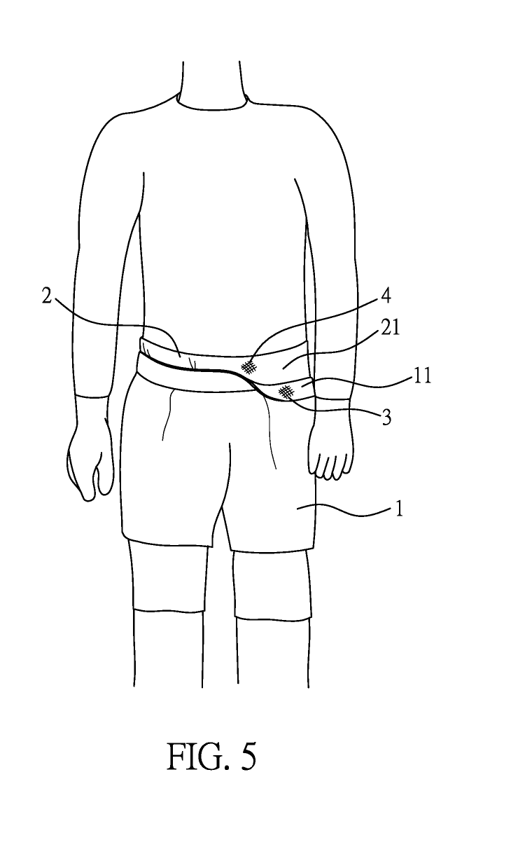

When a user wears the first wearing article 1 and the second wearing article 2 and makes the first engaging effect zone 11 in contact with the second engaging effect zone 21, the tangling-hairs fibers 32 of the first engaging effect zone 11 will be engaged and adhered with the fibriform loops 42 of the second engaging effect zone 21 to prevent a lower part of the sport tight top (the first wearing article 1) from slipping out from inside of the waist of the sport tight pants (the second wearing article 2) when the user exercises his/her body, thereby keeping a tidy outfit. Please refer to FIG. 5, which illustrates a third embodiment of the wearing set of the present invention, including a first wearing article 1, which is a pair of pants, and a second wearing article 2, which is a pair of sport tight leggings. The first wearing article 1 has at least one first engaging effect zone 11 implemented with a tangling-hairs fabric 3 on an inner surface of a waist of the pants. The second wearing article 2 has at least one second engaging effect zone 21 implemented with a textured-loops fabric 4 on an external surface of a waist of the sport tight leggings.

When a user wears the first wearing article 1 and the second wearing article 2 and makes the first engaging effect zone 11 in contact with the second engaging effect zone 21, the tangling-hairs fibers 32 of the first engaging effect zone 11 will be engaged and adhered with the fibriform loops 42 of the second engaging effect zone 21 to prevent the pants (the first wearing article 1) from slipping relative to the leggings (the second wearing article 2) when the user exercises his/her body, thereby keeping a tidy outfit.

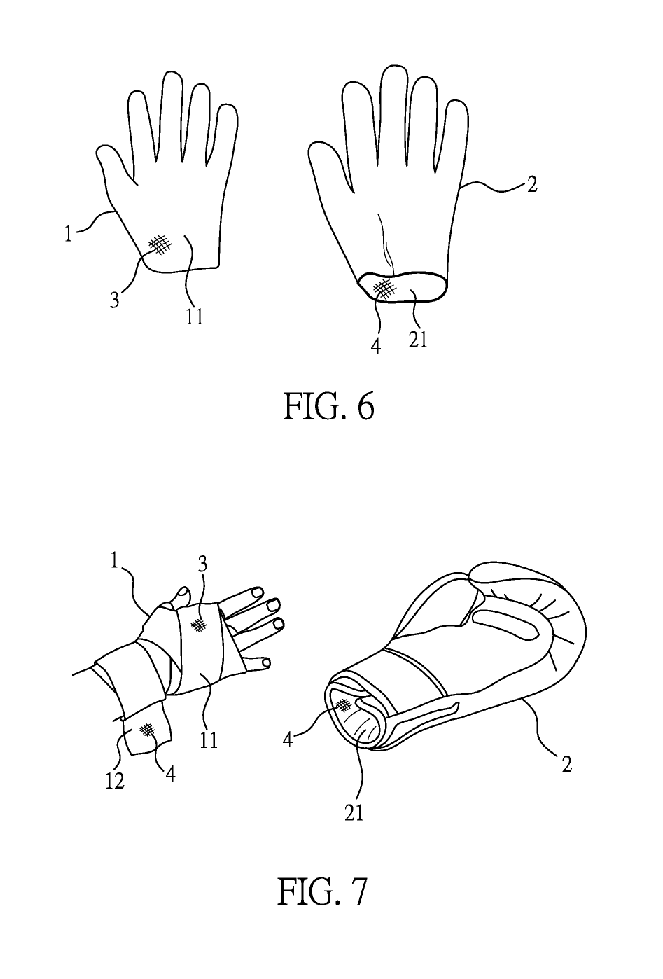

Please refer to FIG. 6, which illustrates a fourth embodiment of the wearing set of the present invention, including a first wearing article 1, which is an inner glove, and a second wearing article 2, which is an outer glove. The first wearing article 1 has at least one first engaging effect zone 11 implemented with a tangling-hairs fabric 3 on an external surface of the inner glove. The second wearing article 2 has at least one second engaging effect zone 21 implemented with a textured-loops fabric 4 on an inner surface of the outer glove.

When a user wears the first wearing article 1 and the second wearing article 2 and makes the first engaging effect zone 11 in contact with the second engaging effect zone 21, the tangling-hairs fibers 32 of the first engaging effect zone 11 will be engaged and adhered with the fibriform loops 42 of the second engaging effect zone 21 to prevent the outer glove (the second wearing article 2) from slipping relative to the inner glove (the first wearing article 1), thereby enabling the user's firmly gripping an object.

Please refer to FIG. 7, which illustrates a fifth embodiment of the wearing set of the present invention, including a first wearing article 1, which is a strip of bandage for wrapping a hand, and a second wearing article 2, which is a boxing glove. The first wearing article 1 has at least one first engaging effect zone 11 implemented with a tangling-hairs fabric 3 on an external surface of the bandage and at least one third engaging effect zone 12 implemented with a textured-loops fabric 4 on an inner surface of the bandage, so that the inner surface can be fastened with the external surface. The second wearing article 2 has at least one second engaging effect zone 21 implemented with a textured-loops fabric 4 on an inner surface of the boxing glove.

When a user wears the first wearing article 1 and the second wearing article 2 and makes the first engaging effect zone 11 in contact with the second engaging effect zone 21, the tangling-hairs fibers 32 of the first engaging effect zone 11 will be engaged and adhered with the fibriform loops 42 of the second engaging effect zone 21 to prevent the boxing glove (the second wearing article 2) from slipping relative to the bandage (the first wearing article 1), thereby ensuring that the boxing glove and the bandage are firmly fastened with each other to make the boxing glove reliably attached with the hand.

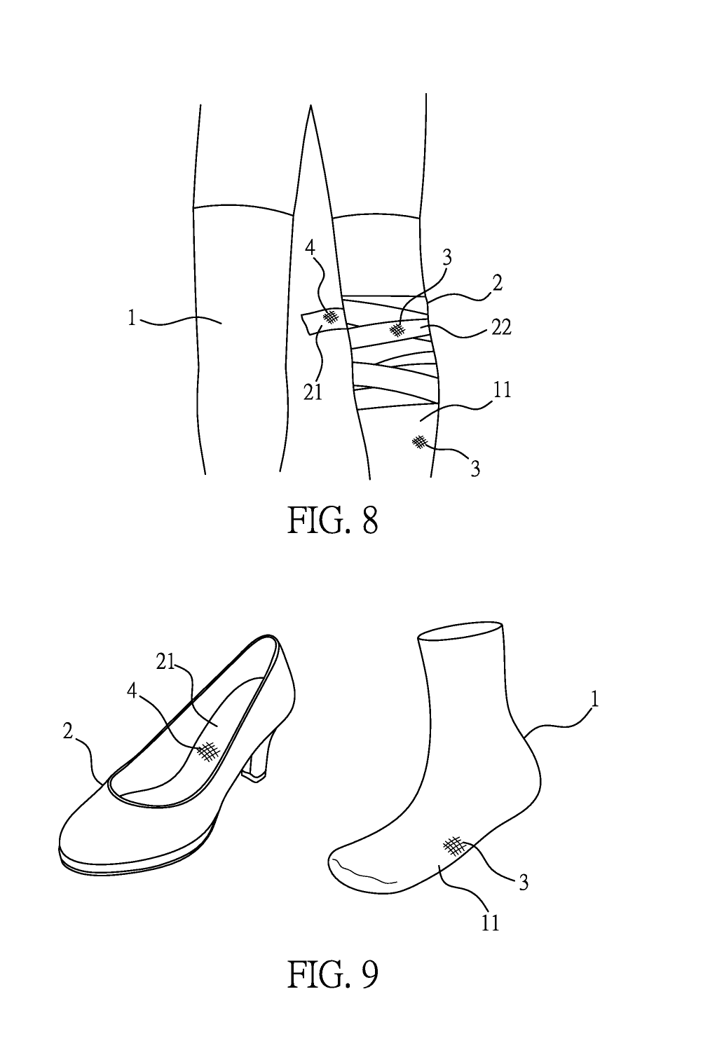

Please refer to FIG. 8, which illustrates a sixth embodiment of the wearing set of the present invention, including a first wearing article 1, which is a tight suite or compression sleeve, and a second wearing article 2, which is a strip of bandage. The first wearing article 1 has at least one first engaging effect zone 11 implemented with a tangling-hairs fabric 3 on an external surface thereof. The second wearing article 2 has at least one second engaging effect zone 21 implemented with a textured-loops fabric 4 on an inner surface of the bandage and at least one third engaging effect zone 22 implemented with a tangling-hairs fabric 3 on an external surface of the bandage, so that the inner surface can be fastened with the external surface.

When a user wears the first wearing article 1 and the second wearing article 2 and makes the first engaging effect zone 11 in contact with the second engaging effect zone 21, the tangling-hairs fibers 32 of the first engaging effect zone 11 will be engaged and adhered with the fibriform loops 42 of the second engaging effect zone 21 to prevent the bandage (the second wearing article 2) from slipping off the tight suite or compression sleeve (the first wearing article 1), thereby providing an adjustable bonding strength. The design is especially helpful for properly compressing a body joint.

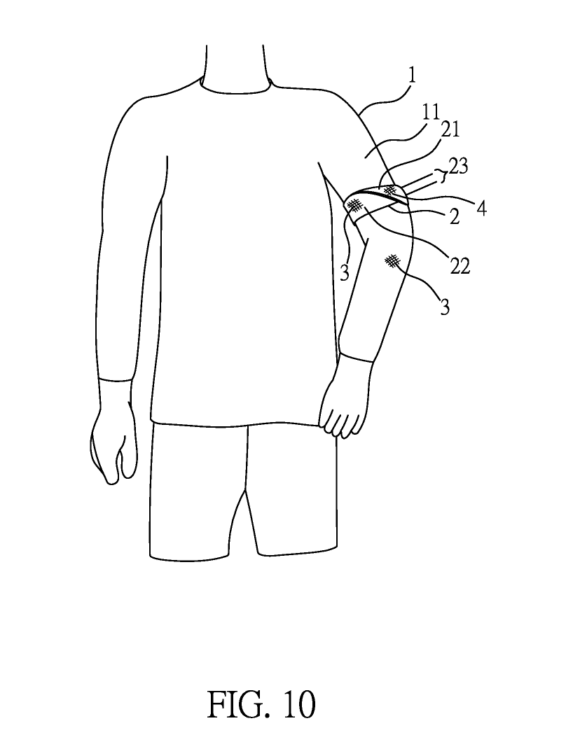

Please refer to FIG. 9, which illustrates a seventh embodiment of the wearing set of the present invention, including a first wearing article 1, which is a pair of socks, and a second wearing article 2, which is a pair of shoes having linings. The first wearing article 1 has at least one first engaging effect zone 11 implemented with a tangling-hairs fabric 3 on an external surface under or around the sole of the socks. The second wearing article 2 has at least one second engaging effect zone 21 implemented with a textured-loops fabric 4 on an exposed surface of the linings.

When a user wears the first wearing article 1 and the second wearing article 2 and makes the first engaging effect zone 11 in contact with the second engaging effect zone 21, the tangling-hairs fibers 32 of the first engaging effect zone 11 will be engaged and adhered with the fibriform loops 42 of the second engaging effect zone 21 to prevent the socks (the second wearing article 1) from slipping relative to the linings (the first wearing article 2), thereby making the shoes moving simultaneously with the foots and therefore providing a comfortable wearing experience.

Please refer to FIG. 10, which illustrates an eighth embodiment of the wearing set of the present invention, including a first wearing article 1, which is an electrically conductive top, and a second wearing article 2, which is a strip of electrically conductive bandage. The first wearing article 1 has at least one first engaging effect zone 11 implemented with an electrically conductive tangling-hairs fabric 3 on an external surface thereof The second wearing article 2 has at least one second engaging effect zone 21 implemented with an electrically conductive textured-loops fabric 4 on an inner surface thereof and at least one third engaging effect zone 22 implemented with an electrically conductive tangling-hairs fabric 3 on an external surface thereof, so that the inner surface can be fastened with the external surface. Besides, a wire 23 can be connected with the second engaging effect zone 21 to electrically connect with the textured-loops fabric 4.

When a user wears the first wearing article 1 and the second wearing article 2 and makes the first engaging effect zone 11 in contact with the second engaging effect zone 21, the tangling-hairs fibers 32 of the first engaging effect zone 11 will be engaged and adhered with the fibriform loops 42 of the second engaging effect zone 21 to prevent the bandage (the second wearing article 2) from slipping off the top (the first wearing article 1), and the bandage (the second wearing article 2) can serve as an electric current path or a switch to connect with a wearing type electronic product.

Similarly, the wearing set can also be applied for heat retaining applications. For example, the first wearing article can be a top, the at least one first engaging effect zone can be located on an external surface of a collar thereof and the second wearing article can be a scarf, with the at least one second engaging effect zone on an inner surface thereof.

Besides, it is to be noted that, in the embodiments from the second embodiment (shown in FIG. 4) to the eighth embodiment (shown in FIG. 10), the first engaging effect zone 11 can be implemented with the textured-loops fabric 4 and the second engaging effect zone 21 can be implemented with the tangling-hairs fabric 3.

Thanks to the designs mentioned above, the present invention can offer advantages as follows:

1. As the tangling-hairs fabric is implemented with meldable fibers, when the tangling-hairs fabric is a single jersey mesh fabric or a double jersey fabric possessing features of having a proper friction and stretchable elasticity, capable of absorbing moisture, getting dry quickly, and providing an antibacterial effect, the tangling-hairs fabric can serve as a fabric material for a sport tight top or sport tight paints, where the tangling-hairs fabric can be engaged and adhered with the textured-loops fabric to prevent overlapping surfaces of the wearing set from sliding relative to each other, while simultaneously possess the advantages of being capable of absorbing moisture, perspiration, and providing air permeability, thereby maintaining a tidy outfit when a wearer is doing an exercise.

The textured-loops fabric can be implemented with a conjugated-fiber yarn, which can go through a fiber-splitting process to form much larger surface areas to provide a moisture absorbing effect and thereby enable the textured-loops fabric to be used for a sport tight top or sport tight pants, where the loop fabric is to be engaged with the tangling-hairs fabric to prevent overlapping surfaces of the wearing set from sliding relative to each other, and thereby maintain a tidy outfit and facilitate a wearer's doing an exercise.

2. When the wearing set includes a tight suit (or a compression sleeve) and a strip of bandage, the present invention is capable of overcoming the problem of conventional bandage being easy to slip out of place, especially when the bandage is wrapped around a joint. Besides, the tangling-hairs fabric implemented on a tight suit or compression sleeve, after being engaged with the textured-loops fabric on the bandage, can be like a skin layer firmly attached with the wearer, thereby greatly promoting the protection function thereof.

In addition, when the wearing set is a two-layer glove, the present invention can provide a proper friction between an inner glove and an outer glove to make them tightly attached to each other to facilitate a wearer to firmly grip an object, and the fastening means will not hurt hands.

3. When the wearing set includes socks and shoe linings, the present invention can provide a proper friction between them to prevent overlapping surfaces thereof from sliding relative to each other, thereby facilitating a wearer's walking.

In addition, when the wearing set includes a top and a scarf, the present invention can provide a proper friction between them to prevent overlapping surfaces thereof from sliding relative to each other, thereby facilitating a wearer's wearing the scarf.

While the invention has been described by way of example and in terms of preferred embodiments, it is to be understood that the invention is not limited thereto.

To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A wearing set capable of preventing overlapped articles from sliding past each other, including:
a first wearing article having at least one first engaging effect zone; and
a second wearing article having at least one second engaging effect zone;
wherein one zone of the first engaging effect zone and the second engaging effect zone has a tangling-hairs fabric and the other zone of the first engaging effect zone and the second engaging effect zone has a textured-loops fabric; the tangling-hairs fabric includes at least one meldable-staple-fibers spun yarn and has a plurality of tangling-hairs fibers protruding outwardly from a surface thereof, the tangling-hairs fibers having curled terminals or ball-shaped terminals formed by a singeing process; the textured-loops fabric includes at least one different-shrinkage-fibers yarn component and has a plurality of fibriform loops on a surface thereof, the fibriform loops being formed by a thermal contraction process; and when the tangling-hairs fabric and the textured-loops fabric are overlapped to contact each other, the fibriform loops will be engaged or adhered with the tangling-hairs fibers to prevent the first wearing article from sliding relative to the second wearing article.

2. A wearing set capable of preventing overlapped articles from sliding past each other, including:
a first wearing article having at least one first engaging effect zone; and
a second wearing article having at least one second engaging effect zone;
wherein one zone of the first engaging effect zone and the second engaging effect zone has a tangling-hairs fabric and the other zone of the first engaging effect zone and the second engaging effect zone has a textured-loops fabric; the tangling-hairs fabric includes at least one meldable-filament-fibers yarn and has a plurality of tangling-hairs fibers on a surface thereof, the tangling-hairs fibers having curled terminals or ball-shaped terminals formed by a peaching process and a singeing process; the textured-loops fabric includes at least one different-shrinkage-fibers yarn component and has a plurality of fibriform loops on a surface thereof, the fibriform loops being formed by a thermal contraction process; and when the tangling-hairs fabric and the textured-loops fabric are overlapped to contact each other, the fibriform loops will be engaged or adhered with the tangling-hairs fibers to prevent the first wearing article from sliding relative to the second wearing article.

3. The wearing set capable of preventing overlapped articles from sliding past each other as disclosed in claim 1, wherein the meldable-staple-fibers yarn includes fibers selected from a group consisting of polyester fibers, polyamide fibers, polyolefin fibers, and polyacrylonitrile fibers.

4. The wearing set capable of preventing overlapped articles from sliding past each other as disclosed in claim 2, wherein the meldable-filament-fibers yarn includes fibers selected from a group consisting of polyester fibers, polyamide fibers, polyolefin fibers, and polyacrylonitrile fibers.

5. The wearing set capable of preventing overlapped articles from sliding past each other as disclosed in claim 1, wherein the tangling-hairs fabric is fabricated by a weaving process, a warp knitting process, or a weft knitting process, and is a fabric selected from a group consisting of a single jersey mesh fabric, a single jersey fabric, a double jersey fabric, a plain fabric, and a twill fabric.

6. The wearing set capable of preventing overlapped articles from sliding past each other as disclosed in claim 2, wherein the tangling-hairs fabric is fabricated by a weaving process, a warp knitting process, or a weft knitting process, and is a fabric selected from a group consisting of a single jersey mesh fabric, a single jersey fabric, a double jersey fabric, a plain fabric, and a twill fabric.

7. The wearing set capable of preventing overlapped articles from sliding past each other as disclosed in claim 1, wherein the textured-loops fabric is fabricated by a weaving process, a warp knitting process, or a weft knitting process, and is a fabric selected from a group consisting of a single jersey mesh fabric, a single jersey fabric, a double jersey fabric, a plain fabric, and a twill fabric.

8. The wearing set capable of preventing overlapped articles from sliding past each other as disclosed in claim 2, wherein the textured-loops fabric is fabricated by a weaving process, a warp knitting process, or a weft knitting process, and is a fabric selected from a group consisting of a single jersey mesh fabric, a single jersey fabric, a double jersey fabric, a plain fabric, and a twill fabric.

9. The wearing set capable of preventing overlapped articles from sliding past each other as disclosed in claim 1, wherein the textured-loops fabric is fabricated by a weaving process, a warp knitting process, or a weft knitting process to form the fibriform loops with protruding loops, and each fiber in the fibriform loops has a size smaller than 1.04 de/f.

10. The wearing set capable of preventing overlapped articles from sliding past each other as disclosed in claim 2, wherein the loop fabric is fabricated by a weaving process, a warp knitting process, or a weft knitting process to form the fibriform loops with protruding pile-loops, and each fiber in the fibriform loops has a size smaller than 1.04 de/f.

11. The wearing set capable of preventing overlapped articles from sliding past each other as disclosed in claim 1, wherein the loop fabric is fabricated by a weaving process, a warp knitting process, or a weft knitting process to form a plain fabric, and each fiber in the fibriform loops of the plain fabric has a size smaller than 1.04 de/f.

12. The wearing set capable of preventing overlapped articles from sliding past each other as disclosed in claim 2, wherein the loop fabric is fabricated by a weaving process, a warp knitting process, or a weft knitting process to form a plain fabric, and each fiber in the fibriform loops of the plain fabric has a size smaller than 1.04 de/f.

13. The wearing set capable of preventing overlapped articles from sliding past each other as disclosed in claim 1, wherein the first wearing article is a top having the at least one first engaging effect zone implemented on an external surface of a lower part of the top, and the second wearing article is a pair of pants having the at least one second engaging effect zone implemented on an internal surface of a waist of the pants.

14. The wearing set capable of preventing overlapped articles from sliding past each other as disclosed in claim 2, wherein the first wearing article is a top having the at least one first engaging effect zone implemented on an external surface of a lower part of the top, and the second wearing article is a pair of pants having the at least one second engaging effect zone implemented on an internal surface of a waist of the pants.

15. The wearing set capable of preventing overlapped articles from sliding past each other as disclosed in claim 1, wherein the first wearing article is a top having the at least one first engaging effect zone implemented on an inner surface of a lower part of the top, and the second wearing article is a pair of pants having the at least one second engaging effect zone implemented on an external surface of a waist of the pants.

16. The wearing set capable of preventing overlapped articles from sliding past each other as disclosed in claim 2, wherein the first wearing article is a top having the at least one first engaging effect zone implemented on an internal surface of a lower part of the top, and the second wearing article is a pair of pants having the at least one second engaging effect zone implemented on an external surface of a waist of the pants.

17. The wearing set capable of preventing overlapped articles from sliding past each other as disclosed in claim 1, wherein the first wearing article is a pair of leggings having the at least one first engaging effect zone implemented on an external surface of a waist of the leggings, and the second wearing article is a pair of pants having the at least one second engaging effect zone implemented on an internal surface of a waist of the pants.

18. The wearing set capable of preventing overlapped articles from sliding past each other as disclosed in claim 2, wherein the first wearing article is a pair of leggings having the at least one first engaging effect zone implemented on an external surface of a waist of the leggings, and the second wearing article is a pair of pants having the at least one second engaging effect zone implemented on an internal surface of a waist of the pants.

19. The wearing set capable of preventing overlapped articles from sliding past each other as disclosed in claim 1, wherein the first wearing article is an inner glove having the at least one first engaging effect zone implemented on an external surface thereof, and the second wearing article is an outer glove having the at least one second engaging effect zone implemented on an internal surface thereof.

20. The wearing set capable of preventing overlapped articles from sliding past each other as disclosed in claim 2, wherein the first wearing article is an inner glove having the at least one first engaging effect zone implemented on an external surface thereof, and the second wearing article is an outer glove having the at least one second engaging effect zone implemented on an internal surface thereof.

21. The wearing set capable of preventing overlapped articles from sliding past each other as disclosed in claim 1, wherein the first wearing article is a pair of socks each having the at least one first engaging effect zone implemented on an external surface thereof, and the second wearing article is a pair of shoe linings each having the at least one second engaging effect zone implemented on a surface thereof.

22. The wearing set capable of preventing overlapped articles from sliding past each other as disclosed in claim 2, wherein the first wearing article is a pair of socks each having the at least one first engaging effect zone implemented on an external surface thereof, and the second wearing article is a pair of shoe linings each having the at least one second engaging effect zone implemented on a surface thereof.

23. The wearing set capable of preventing overlapped articles from sliding past each other as disclosed in claim 1, wherein the first wearing article is a strip of bandage for wrapping a hand, the strip of bandage having the at least one first engaging effect zone implemented on an external surface thereof, and the second wearing article is a boxing glove having the at least one second engaging effect zone implemented on an inner surface thereof.

24. The wearing set capable of preventing overlapped articles from sliding past each other as disclosed in claim 2, wherein the first wearing article is a strip of bandage for wrapping a hand, the strip of bandage having the at least one first engaging effect zone implemented on an external surface thereof, and the second wearing article is a boxing glove having the at least one second engaging effect zone implemented on an inner surface thereof.

25. The wearing set capable of preventing overlapped articles from sliding past each other as disclosed in claim 23, wherein the strip of bandage further has at least one third engaging effect zone implemented on an inner surface thereof, the at least one third engaging effect zone being capable engaging and adhering with the at least one first engaging zone to make the inner surface fastened with the external surface of the strip of bandage.

26. The wearing set capable of preventing overlapped articles from sliding past each other as disclosed in claim 24, wherein the strip of bandage further has at least one third engaging effect zone implemented on an inner surface thereof, the at least one third engaging effect zone being capable of engaging and adhering with the at least one first engaging zone to make the inner surface fastened with the external surface of the strip of bandage.

27. The wearing set capable of preventing overlapped articles from sliding past each other as disclosed in claim 1, wherein the first wearing article is a tight suit having the at least one first engaging effect zone implemented on an external surface thereof or a compression sleeve having the at least one first engaging effect zone implemented on an external surface thereof, and the second wearing article is a strip of bandage having the at least one second engaging effect zone implemented on an inner surface thereof.

28. The wearing set capable of preventing overlapped articles from sliding past each other as disclosed in claim 2, wherein the first wearing article is a tight suit having the at least one first engaging effect zone implemented on an external surface thereof or a compression sleeve having the at least one first engaging effect zone implemented on an external surface thereof, and the second wearing article is a strip of bandage having the at least one second engaging effect zone implemented on an inner surface thereof.
